(19) United States
(12) Patent Application Publication
(10) Pub. No.: US 2002/0119470 A1
(43) Pub. Date: Aug. 29, 2002

(54) Magnetic bead-based array for genetic detection

(76) Inventors: Michael I. Nerenberg, La Jolla, CA (US); Geoffrey C. Landis, Carlsbad, CA (US); Lorelei P. Westin, La Mesa, CA (US); James P. O'Connell, Solana Beach, CA (US); Ling Wang, San Diego, CA (US); Ray R. Radtkey, San Diego, CA (US); Lana L. Feng, Del Mar, CA (US)

Correspondence Address: LYON & LYON LLP, 633 WEST FIFTH STREET, SUITE 4700, LOS ANGELES, CA, 90071, US

(21) Appl. No.: 09/972,830

(22) Filed: Oct. 5, 2001

Related U.S. Application Data
(63) Continuation of application No. PCT/US00/10121, filed on Apr. 12, 2000.
(60) Provisional application No. 60/129,389, filed on Apr. 13, 1999.

(51) Int. Cl. C12Q 1/68; C12M 1/34

(52) U.S. Cl. 435/6; 435/287.2

(57) ABSTRACT

This invention provides a bead array counter system that combines strand displacement amplification with magnetoresistive micro sensor chips and magnetic beads. The system allows for detection of target nucleic acids in highly dilute samples. The system further provides a means to detect specific nucleic acid sequences comprising SNPs and STRs.

### FIELD OF THE INVENTION

This invention relates to detection of molecules in a sample using a bead array counter type device. More specifically, this invention relates to augmenting the performance of detection of target nucleic acid molecules in test samples using a combination of anchored strand displacement amplification (aSDA) on the surface of a magnetic bead and magnetoresistive sensor arrays. Additionally, this invention facilitates interaction of large volume samples to micro detecting formats of a bead array device.

### BACKGROUND SUMMARY

The following description provides a summary of information relevant to the present invention. It is not an admission that any of the information provided herein is prior art to the presently claimed invention, nor that any of the publications specifically or implicitly referenced are prior art to that invention.

In 1994, researchers at the Naval Research Laboratory (NRL) covalently attached single-stranded DNA probes to the cantilever-beam force transducer of an atomic force microscope (AFM) and to a silicon substrate. The cantilever and substrate were brought together in the presence of longer, free-floating “target” DNA that hybridized to the probes of the AFM and substrate. The experiment was designed such that there would be an average of one target nucleic acid strand hybridized to the probes connecting the cantilever to the substrate. The cantilever was then pulled away from the substrate, placing increasing tension on the hybridization bonds between the target and probe molecules until the hybridizing strands were pulled apart. By observing the sudden drop in force (tension) that occurred when the hybridizing bonds broke, the researchers were able to detect and characterize individual target molecules.

In recent years the NRL has replaced cantilever and substrates with magnetic beads and biosensors in order to test the properties of hybrizidations of target molecules and probes. In this modern methodology, characterization of hybridizing molecules is carried out in part by magnetically pulling the bound beads with a known controlled small magnetic force. The strength of the hybridization is tested by observing whether the beads detach from the sensor surface due to such force. Unbound and non-specifically bound beads may be readily removed from the sensor surface while use of larger forces can be used to break intermolecular bonds and thereby characterize the strength of molecular interactions.

We have developed an advance in the art of such small force detection using a bead array counter (BARC) which combines magnetic beads and anchored strand displacement amplification with giant magnetoresistive-sensing (GMR-sensing) microscope technology to detect biomolecules with single-molecule sensitivity.

### SUMMARY OF THE INVENTION

According to the embodiments of the invention, a microfabricated detector system comprising various components is provided. In a first embodiment, the system comprises single-component sensors having magnetoresistive qualities. These sensors are micron-sized and provide a substrate to which probe molecules, such as natural and/or synthetic nucleic acids, and/or proteins such as antibodies, receptors, enzymes etc., are attached.

In one embodiment, the probes attached to the sensors are capable of participating in amplification reactions, particularly strand displacement amplification reactions. In this embodiment, the sensors can be used to attract target nucleotides for amplification followed by detection of the amplified species using probe-labeled magnetic beads.

In another embodiment the invention contemplates use of magnetic beads having a second probe capable of participating in amplification reactions. The beads may have attached thereto anywhere from one to a multiplicity of probes.

In another embodiment, the system is capable of detecting target molecules of interest in test samples. Such target molecules are contemplated to include nucleic acids, polypeptides, and/or organic molecules. Where target molecules are contemplated to be nucleic acids, in a preferred embodiment, the probes attached to the beads and sensor substrate are designed in part to be complementary to the target nucleic acid sequences.

In another embodiment, the system contemplates employment of anchored SDA of the targets which will provide for an increased population of sought for target molecules in the form of amplicons attached directly to either the sensors or the beads. It is contemplated that where the amplicon is formed on the beads, the amplicon-bearing beads may be brought into proximity of the sensor array and allowed to participate in hybridization of the distal end of the target amplicon with the probe of the sensor substrate. Alternatively, where the amplicon is formed on the sensors, the probe-bearing beads may be brought into proximity of the sensor for hybridization. In either case, following such hybridization controlled magnetic forces may be employed to remove non-specifically bound beads and to test hybridization characteristics of the target species.

In a particularly preferred embodiment, the BARC system uses controlled forces in the manner of an AFM to distinguish differences between specific and nonspecific hybridization interactions between the capture probe and target molecule. This allows for high sensitivity and selectivity per unit of detector area in detecting the presence of hybridization events. Such sensitivity is dependent on the size of the detector. This is because larger detectors collect more target molecules resulting in attachment of more magnetic beads which in turn provides for greater sensitivity at lower concentrations of target. In one embodiment, the sensitivity provides for detection of at least 1000 different analytes detected at 20,000 copy/ml.

In a further embodiment, the sensors contemplated for the system of the invention detect a magnetic field produced by the attached beads and can determine the exact number of beads so attached. We therefore refer to this device and system as the anchored SDA bead array counter or aSDA/BARC. This device and system can be used to simultaneously monitor hundreds, or even thousands, of analytes.

In another embodiment, the BARC system of the invention is used to assay target molecules of interest in liquid or flowable medium samples. Generally, the BARC device provides a platform for carrying out immunoassays, drug-target interaction assays, or any other type of binding assay. The specific nature of the assay will simply depend upon the type of probe used and target molecule sought for detection.

In yet another embodiment, the BARC system of the invention is amenable to recycling of its sensor components which may be reprogrammed with new specified probes of interest, for example where the sensor has applied thereto capture probes, the sensor may be freed of such probes by washing the sensor device at 94° C. to remove nucleic acids attached thereto.

In still other embodiments, the BARC system of the invention provides for multiple-analyte analysis in a portable format for detection, characterization, and containment of human, animal, and plant pathogens as well as discovery of drug candidates.

### BRIEF DESCRIPTION OF THE DRAWINGS

FIG. 1 shows a design schematic for a BARC sensor chip for use in assaying target molecules of interest according to the invention. Depicted is a GMR sensor chip 1 with magnetic beads containing either non-specific 6 or specific probe/amplicons 5. The magnetic beads hybridize to the surface of the GMR sensor array 2 through attached nucleic acid probes at the surface of the microchip 3. Alternatively, the probe-bearing beads may be hybridized to specific probe/amplicons attached to the sensor. Magnetic beads containing non-specifically bound molecules fail to hybridize to the sensor 4 whether the sensor is amplicon-labeled or simply probe-labeled. Thus, only specific products are counted on the GMR sensor array.

FIG. 2 shows a scheme for the integration of anchored SDA with BARC detection. Stage 11 shows a mixed bead population (e.g., in the example of the figure, beads specific to 3 different targets). Each bead of a particular probe population comprises both sense and antisense primers (attached covalently or via streptavidin-biotin) 9, which are specific for a particular target sequence. The probes so attached provide for the ability for SDA to take place directly on each population of beads. Next, the beads are placed in a thermally controlled chamber 8 which contains dried lysis buffer. The sample (e.g., blood) 7 is added and lysis/denaturation of nucleic acid 10 is followed by hybridization of target molecules to the beads 12. Extraneous material is washed away and complete SDA mix (containing buffers, nucleotides and enzymes) is added 13. Amplification generates anchored double stranded amplicons 14. Heat denaturation leaves only one strand anchored to the bead 15. The beads are then placed on the BARC chip for hybridization 16.

FIGS. 3A, B, and C show an example of detection of target nucleotides using anchored SDA on beads and microchip arrays. In this case, the specific beads were identified using fluorescent technology. Magnetic beads were coated with amplification primers for two sets of bacterial genes: Yst and SLTI. Target DNA was denatured and added to the magnetic beads 17, and anchored SDA initiated as in FIG. 2. Control beads 18 had no target DNA added to the reaction. The double-stranded amplicons anchored onto the bead surface were denatured and both the amplified and control beads were electronically addressed to the microchip array 19. A white light image 20 (3A) was taken to show that both the amplified and control beads addressed equally to the microchip array. However, when specific reporters for either Yst 21 or SLT I 22 (3B and 3C respectively) were added, only the beads containing amplified target exhibited any fluorescent signal, confirming that amplification was accomplished on the bead surface in a target-specific manner.

### DETAILED DESCRIPTION OF THE PREFERRED EMBODIMENTS

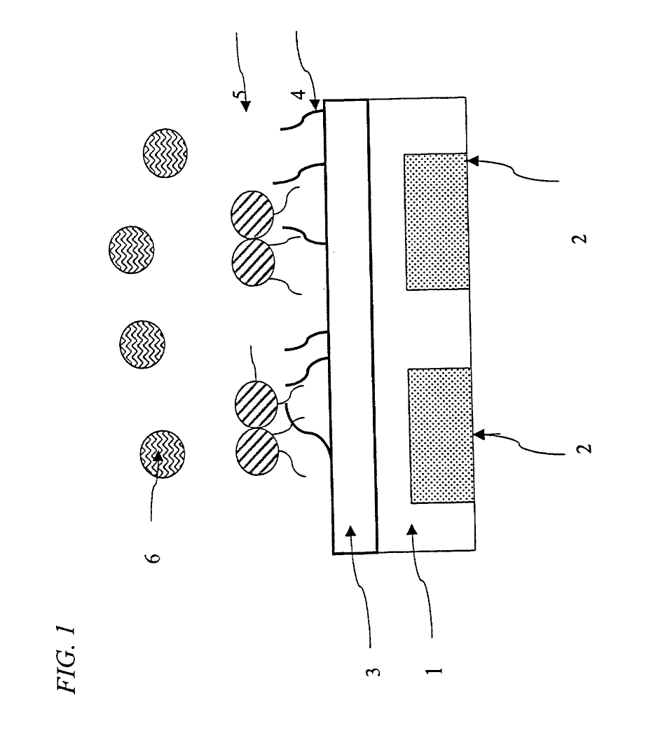

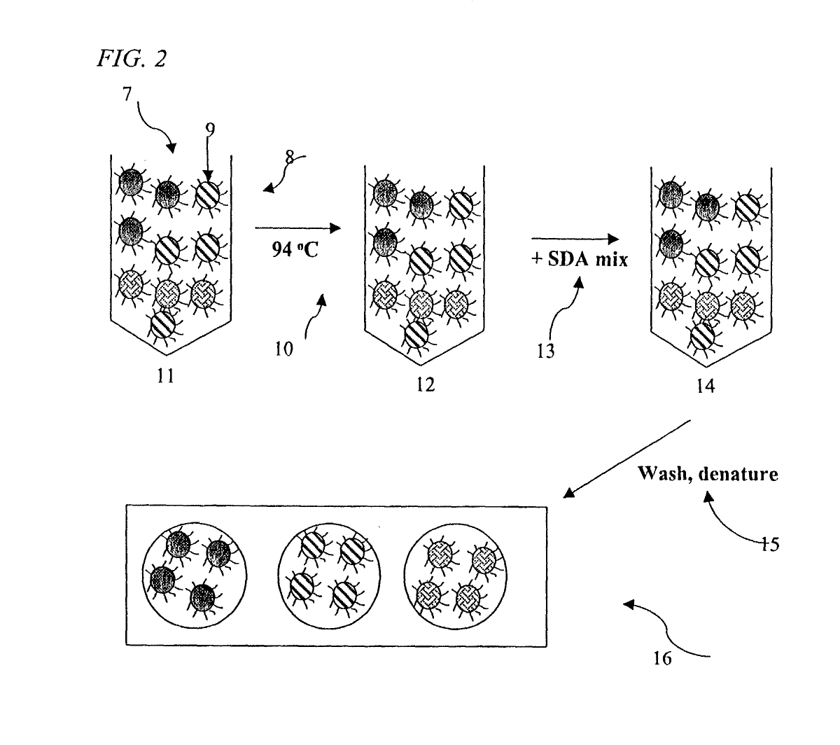

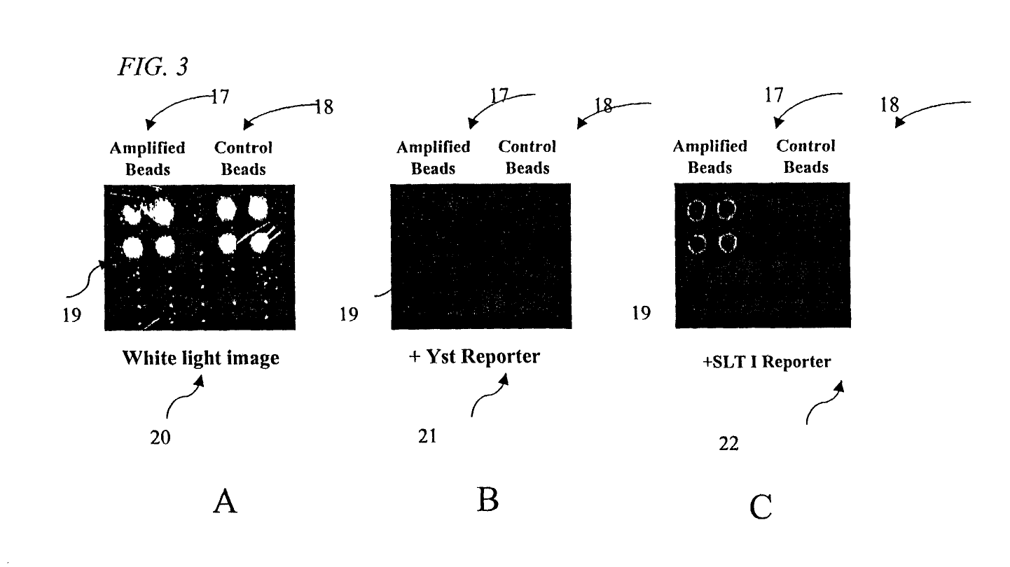

As will be understood by one of skill in the relevant art, the BARC device and system of the invention allows for the detection of nucleic acids of interest from a flowable medium sample by detecting the binding of target nucleic acid either by amplifying the target directly on the bead or magnetoresistive sensor. The target molecule must be first captured by probes that are attached to the surface of the sensor or magnetic beads (as shown in FIG. 1). In a preferred embodiment of the BARC system, the system employs the use of aSDA for increasing the population of sought for tar-get molecules as depicted in FIG. 2. The use of aSDA provides for highly-effective biosensor sampling of target molecules of interest because of the presence, following aSDA, of large relative amounts of target molecules as compared to non-target molecules as demonstrated in FIG. 3. The combination of aSDA and BARC sensors is preferable as sample detection may be carried out in a minimal time frame (minutes to seconds).

The current system is much advanced over existing AFM cantilever detection schemes in that rather than bridging between a substrate and an AFM tip, the target nucleic acid bridges a substrate sensor and a magnetic microbead. The magnetic quality of the bead can be used to create the tension force necessary to bring about disassociation of the hybridized complex, i.e., if the hybridization bond breaks, the bead is pulled away from the substrate. In a further preferred embodiment, such a format allows for testing of bond strengths by determining whether the bead is still present after exerting a known force to separate the bead from the sensor. This will allow for discrimination of hybridization strengths present, and give valuable information related to genotyping or identifying nucleic acid samples.

In one format of the BARC device and system, a multiplicity of magnetic beads can be brought into proximity with a magnetoresistive substrate sensor with each bead subjected to the same magnetic force. Once the beads and sensor substrate are in proximity to one another, and in the presence of a flowable medium containing target molecules of interest, conditions are applied to the system to facilitate binding of the targets to their respective amplification probes (whether bead or sensor bound). After binding, the amplification reaction is performed followed by hybridization of the beads (probe-bearing or amplicon-bearing) to the sensors (amplicon-bearing or probe bearing). The system is then programmed to carry outforce discrimination by applying a known magnetic force to the population of beads as is understood by one of skill in the art. If binding has occurred, then detection of the occurrence of binding is determined by counting the number of beads remaining on the substrate surface.

This system has applications in infectious disease and environmental testing which often require processing of large volumes of fluid materials to detect the presence of target molecules. The system of the current invention is advantageous by avoiding the need for extensive preprocessing or concentration. Moreover, the system is applicable to processing and testing for target molecules in large sample volumes.

In other advantages, the system allows for direct capture and immobilization of the target species to the surface of the bead or sensor which facilitates sample preparation. For example, cellular lysis, if necessary, and preparation may be done directly in the presence of chaotropic detergent. Second, with respect to detection of nucleic acid targets, the target species may be concentrated from the high volume samples. This is carried out due to the capture of the low level target molecules onto the beads or sensor, each of which may be physically removed from the high volume sample. The isolated, concentrated target-bearing beads or target-bearing sensors may then be subjected to an exponential amplification process. This does two things: (1) it enormously increases the concentration of the target, greatly accelerating kinetics of hybridization, and (2) it reduces the genetic complexity of the target by creating short amplicons for targeting (typically less than 200 bp). The amplification of defined target species provides for the specific design of the moieties of the target molecule involved in hybridizing to the bead-bound and sensor-bound probes. Amplification also allows selective retention of one strand, facilitating separation of individual strands of the amplicon. Such factors augment hybridization to the probe sequences. By virtue of concentration of the target on the bead or sensor, amplification, and the intrinsic sensitivity of the electronic detection of the BARC sensor system, targets present in low concentration should be capable of being detected at the level of 10⁴ targets.

In a preferred embodiment, such amplification may be carried out using anchored strand displacement amplification (aSDA). The methodology of the present invention is further advantageous in that it allows for multiplexing which can be accomplished by using a mixed population of beads wherein different beads within the population harbor different probes capable of participating in SDA for differing targets or that are capable of simply hybridizing to the target. Additionally, such a solid based amplification system will prevent the primer/probes from interfering with each other. Based on previous experience, we believe that a minimum of 20 or more reactions of short amplicons can be readily multiplexed efficiently and reproducibly.

In yet another embodiment, the probe/primers attached to the beads or sensor will comprise a mixture of probes such that although all of the probes will be capable of participating in at least one amplification reaction step, some will be designed so that nicking (restriction enzyme-mediated), which is necessary in at least one step of SDA, is not possible for some primer/probes while it remains possible for others. With the appropriate ratio of cleavable to non-cleavable probes, in a preferred embodiment at least ½ of the amplicons generated will remain covalently attached to the bead by its 5′ end. Unilateral covalent attachment of the noncleavable primers will insure complete strand separation and easy removal of extraneous DNA following simple heat denaturation while washing in water. Once subjected to amplification, the beads containing either target sequence, or capture probe sequence may be sampled by directing the target- or probe-containing beads past the BARC sensor chip. FIG. 2 depicts one example of how SDA is integrated with the BARC system of the invention.

Such a system is a substantial improvement over prior methods that merely applied magnetic beads to a substrate surface, such as a microtiter well, and counted the beads remaining bound following application of magnetic force. For example, force differentiation assays (FDA), have been used to develop the covalent immobilization and antifouling chemistry necessary to perform force discrimination. Previous experiments with FDA for ovalbumin were performed wherein 200-500 magnetic beads were allowed to settle within the field of view of a microscope. If no ovalbumin (the target molecule) is present (A), about 98% of these beads are removed when we apply 1 pN of magnetic force per bead. When ovalbumin is present, there is a noticeable increase in the number of beads remaining bound to the surface under the same magnetic force. Therefore, the 1 pN of force that is generated per bead allows us to effectively discriminate between bound and unbound magnetic particles. In such a system there is a 2% nonspecific binding background that limits sensitivity to 100 pg/ml.

In contrast, such nonspecific binding is greatly reduced in the present BARC system, which due to improvements in various surface chemistries and amplification, among other things, allows for the use of increased application of magnetic force resulting in a further reduction of background. With the application of sufficient magnetic forces, magnetic microbead assays of the current invention possess at least two potential advantages over other hybridization assays. First, they can be used to directly measure bond strengths between the hybridized species. Second, they can achieve extremely high sensitivity.

The sensitivity of hybridization assays and immunoassays is typically limited by 1) nonspecific binding of the label to the sensor and 2) limited sensitivity of the sensor to the presence of label. Force discrimination allows the removal of nonspecifically-bound label using well-controlled magnetic forces. Furthermore, the BARC system possesses a sensitivity that allows detection of single labeled magnetic bead, and therefore a single analyte molecule.

In a further embodiment, the BARC system uses microfabricated magnetic field sensors made of magnetoresistive materials that have high sensitivity and micrometer-scale size. Magnetoresistive materials contemplated for the invention are typically thin-film metal multilayers, the resistance of which changes in response to magnetic fields. Examples of such materials include anisotropic magnetoresistive (AMR) and giant magnetoresistive (GMR) materials.

In a further example of a BARC system assay, biotinylated probe nucleic acid or protein molecules are added to a sample containing target molecules of interest. The probe molecules bind or hybridize with any target molecule present in the sample. Streptavidin-conjugated magnetic beads ˜3 μm in diameter are then introduced to the test sample. These beads bind the probe and following such binding are isolated from the sample by applying a magnetic field. The beads are then resuspended and injected into the BARC device. Within the device the bead suspension is passed through a flow cell that contains a sensor substrate comprising a multiplicity of microfabricated magnetoresistive elements coated on at least one side. The sensor may generally comprise a wafer about 0.5-1 cm² that has a thin insulating or permeation layer overlying the sensors. To the surface of the insulating layer is applied probes for binding target molecules of interest. (Alternatively, the sensors could have amplified target molecules.)

The sensor-containing wafer acts as a detector chip that can detect the presence of magnetic beads that are associated with the sensor due to the binding of probes to target molecules. After using magnetic force to test the strength of the binding of the beads, and to remove weakly adhering beads, the detector chip is used to count the number of remaining beads, which number is proportional to the concentration of target DNA in the sample.

In another example of the BARC system of the invention, prototype arrays were fabricated (FIG. 1) and tested for their ability to detect the binding of magnetic particles by force discrimination. The prototype BARC device is intended for scissoring mode detection, such as depicted in U.S. Pat. No. 5,981,297, herein incorporated by reference, in which a detection field H perpendicular to the plane of the sensor causes the magnetic beads to generate a smaller field B in the plane of the sensor. The sensor, which is sensitive only to in-plane fields, generates a signal roughly proportional to the number of magnetic particles present.

In yet another embodiment, the BARC system of the current invention can accommodate a multiplicity of analytes. The number of analytes that are possible is related to the total active area of the sensor chip (i.e. the area of all of the magnetoresistive sensors together) divided by the amount of area required for each analyte. This is in part dependent upon the amount of space each magnetic bead occupies on the sensor chip. In a preferred embodiment, each square millimeter of substrate will accommodate at least 5,000 2.8 μm Dynabeads. In a further preferred embodiment, at least 100 beads per probe site are used to obtain chemical concentration measurements having acceptable assay-to-assay variability. In yet a further preferred embodiment, the active area per probe is at least 20,000 μm². In such case, use of two probes per analyte (or two redundant sites per probe) on a 1×1 cm sensor chip having an area which is 40% occupied by sensors can accommodate at least 1,000 analytes. This number increases if smaller beads are used since more beads can then be applied per unit area of substrate.

In yet another embodiment, increasing the amount of area per probe improves assay reliability and sensitivity by allowing sampling of a larger population of beads thereby reducing assay-to-assay variability of bead count. Where false positives or negatives are of concern, the number of analytes is reduced and the active area per analyte increased. As an example of applying use of the BARC system to various types of analyte detection, environmental or clinical monitoring applications, in a panel for detecting specific pathogens for instance, the most significant pathogens could be detected at 1300 copy/ml sensitivity, while pathogens of lesser significance could be assayed at 33,000 copy/ml sensitivity. The balance between number of analytes and area per analyte can be tuned without redesigning the sensor chip.

In another embodiment, the BARC system uses magnetic beads such as those currently available from commercial suppliers (Sera-Mag beads, SeraDyn, Inc., and Dynabeads, Dynal, Inc.). Typically, these beads are micrometer-sized particles of iron oxide dispersed in, layered onto, or coated with a polymer or silica matrix to form beads about 1 μm in diameter. These iron-oxide particles are only magnetic in the presence of a magnetic field. Thus, the particles immediately demagnetize when the field is removed, and the beads do not magnetically attract each other and agglomerate. Since iron oxide is not a highly magnetic material, beads containing iron oxide are not practical for use in exerting more than about 5 pN of force per bead. Even with this level of force, force discrimination using the method of the invention is 98% effective such that 2% of beads remain nonspecifically bound to the surface after applying magnetic force.

However, a 5 pN level of force is not enough to break intermolecular bonds which capability is necessary to measure bonds between specific binding pairs, e.g., nucleic acid-nucleic acid (i.e., DNA-DNA DNA-RNA, RNA-RNA, DNA-PNA hybridization), antibody-antigen, or drug-target bonds for example. For drug development applications, the ability to break such noncovalent bonding provides the unique ability to rapidly measure the interaction strength of hundreds of potential compounds with a target molecule on a single sensor chip. For environmental and clinical sensing applications, the ability to quantify bond strength will significantly improve discrimination between specific and nonspecific binding, and therefore allow the high sensitivity and/or high numbers of analytes per chip.

In another embodiment, BARC sensors are constructed with GMR material tailored for use in magnetic field sensors such as handheld Gaussmeters. The signal-to-noise of this material is such that achieving single-magnetic-bead sensitivity requires signal-averaging for about ten seconds. In one embodiment, the detection electronics uses four parallel detection circuits so that 64 sensors can be read in 64×10/4=160 seconds. In another embodiment, a BARC sensor chip having 4096 sensors per chip and which requires significantly higher signal-to-noise, may be constructed for application in the BARC system of the invention by virtue of greatly increased signal levels that are possible with detection methods of the system. Such a sensor allows reduced detection time from 10 seconds to 10 milliseconds. With such a material, a 4096-sensor chip may be read in 10 seconds.

In a preferred embodiment, pseudo spin-valve (PSV) materials are chosen for construction of sensor chips. Materials such as these exhibit the sudden transitions or sharp discontinuity in their response curves. Such a transition is important because the sensor's response to a magnetic bead, or signal per bead, is proportional to the second derivative of the GMR response curve and can be estimated from a GMR response curve that shows how the sensor responds to a magnetic field along its X axis in the absence of magnetic beads.

In still another embodiment, the BARC system uses a fully-automated fluidics system. In a preferred embodiment, the fluidics system comprises a thermoplastic-molded structure having millimeter-scale reservoirs, channels, pumps, and valves. In one embodiment, these components are incorporated into disposable fluidic cartridges that also contain the BARC sensor chip. This fluid dynamics design can evenly and reproducibly disperse magnetic microbeads over the surface of the BARC sensor chip. Since magnetic beads that are useful with the BARC system of the present invention may possess unique qualities, e.g., greater weight than typical magnetic beads, the fluidics system requires such elements as valveless pumps that are based on a diffuser-nozzle design. Such a design does not have magnetic components that might attract magnetic particles, nor does it have mechanical checkvalves that might become clogged by the particles. To control the flow of fluid at channel junctions, clog-proof valves may be employed by using off-cartridge shape memory alloy actuators to pinch off particular channels. Piezoceramic mixing elements can be used to keep beads suspended in solution.

Additionally, cartridge fluidics channels can be mined into plastic substrates, followed by press-molding of diffuser-nozzle elements with a metal mold-pin by technology well understood in the art. In one embodiment, the system uses a pump capable of achieving at least 150 μl/min flow rates with an actuation frequency of at least 700 Hz. In a further preferred embodiment, the miniature actuators used in connection with the fluidics cell should include mechanical amplification of the piezoceramic movement to ensure sufficient compression of the cartridge pump diaphragm as well as independent suspension for each SMA valve actuator to ensure a solid interface with the membrane valve on the cartridge. In a preferred embodiment, SMA valve actuators are able to retract nearly instantaneously upon application of about 1 V at 250 mA power.

In another embodiment, the sensor may incorporate the use of a magnet to sweep the nonspecifically bound beads from the sensor surface. In order to achieve appropriate magnetic force for this purpose, the magnet is preferably designed so as to sweep the sensor at a distance of about 1-2 mm above the sensor surface.

In a further embodiment, signal drift is reduced by use of mounts for the sensor relative to the magnet that avoid variation in sensor output. Variation can occur due to micron-scale movements of the sensor caused by small differences in electromagnetic alignment of the sensor to the magnet, i.e., not perfectly perpendicular to the plane of the GMR sensors. The polarizing field thus causes the sensors to produce a signal that varies with their position relative to the electromagnetic field.

The BARC system has the capability of assessing various diagnostic targets such as determination of SNPs, and STRs for disease and forensics applications. For example, this system may be used to perform as a point of care instrument for determining genetic identity (as might for example, be used for portable database entry and comparison of felons), for doctor's office screening, of genetic mutations, or for identification of agents of infectious disease. As such, it would be suitable for analysis of simple specimens such as blood, buccal swabs, cervical swab, or for culture confirmation from a blood bottle.

By coupling target capture with amplification and detection, the system may be made very sensitive. Given the fact that the beads can be agitated or the sample flowed through, the greatest-potential-may be in analyzing large volumes of dilute fluid. Further, the use of detergent in the hybridization buffer could greatly simplify the stage of sample prep to amplification. These are difficult task for most currently available genetic testing devices and there is a high potential commercial demand for this. Examples of such applications include testing of waste water contamination, diagnosis of sexually transmitted diseases from urine samples, identification of cancerous cells in a fluid aspiration, or processing of forensic samples from a crime scene.

In still other embodiments, the BARC system utilizes both noncovalent and covalent forms of binding of capture and amplification probes to the beads and sensor surfaces. In one scheme, biotinylated DNA capture molecules are attached to streptavidin molecules applied on the beads as shown in Table I. The positive charges in the coating and on the streptavidin molecule will be neutralized with acetic acid N-hydroxysuccinimide ester (AcONSu). Polymer coatings other than Dextran or PEG can be used to further reduce nonspecific binding.

TABLE I

| | |
|---|---|
| (1) | B-NH₂ + SA-COOH -> B-CO₂NH-SA |
| (2) | B-NHCO₂-SA + biotin-DNA --> B-CO₂NH-SA- |
| | biotin-DNA |
| (3) | B-NH₂ + AcONSu -> B-NHAc |

Attachment of oligonucleotides to magnetic beads with capping of free amino groups.
The B = beads and SA = streptavidin.

Chemical schemes to place DNA in specific areas and reduce the positive charges on the remaining surface of the sensor chip are possible via covalent bonding. The synthesis of the derivatized sensor chip is shown in Table II. As shown, N-(2-aminoethyl)-3-aminopropyl-trimethoxylsilane (AEAPS) is chemically adsorbed to the surface of the sensor to functionalize the surface with primary amines. Then the heterobifunctional polyethylene glycols (PEGs)(one end derivatized with a carboxylic acid and the other end functionalized with a protected amino group) is attached to the AEAPS using carbodiimide chemistry in a pH 8.5 buffered solution. The protecting group [t-butoxycarbonyl (BOC) or fluoronyl butoxylcarbonyl (Fmoc)] is removed from the amino group on the PEG, which is attached to the BARC chip. Then an oligonucleotide containing a 3′ carboxylic acid is microdropped over the individual GMR detector areas and coupled according to the conditions described above for amide bond formation. After all of the oligonucleotides are placed on the chip, the unreacted amines on the chip are capped with a pH 8.5 buffered solution containing acetic acid N-hydroxysuccinimide ester.

TABLE II

| | |
|---|---|
| (1) | BC-Si + AEAPS → BC-NH₂ |
| (2) | BC-NH₂ +HOOC-PEG-NHFmoc → BC-HNO₂C-PEG-NHFmoc |
| (3) | BC-HNO₂C-PEG-NHFmoc → BC-HNO₂C-PEG-NH₂ |
| (4) | BC-HNO₂C-PEG-NH₂ + HOOC-3′-DNA-5′-OH → BC-HNO₂C-PEG-HNO₂C- |
| | 3′-DNA5′-OH |
| (5) | BC-NH₂ + BC-NHO₂C-PEG-NH₂ + AcONSu → BC-HNAc + BC-HNO₂C-PEG- |
| | HNAc |

Attachment of DNA polymer on sensor chip with capping of the free amino groups. The BC = sensor chip, Si = silyl group, AEAPS = N-(2-aminoethyl)-3aminopropyl-trimethoxylsilane, DNA = deoxynucleic acid, PEG = poly-ethyleneglycol,

Different types of coupling chemistries, reactive functional groups, and polymer chains may be used in order to reduce the charge-to-charge interactions between the derivatized beads and the derivatized surface as would be understood by those skilled in the art.

As described above, the system of the invention is an integrated genetic analysis system that integrates the following process steps without user intervention or potential for contamination; cell lysis, nucleic acid amplification (where appropriate), nucleic acid identification (or identification of other target molecules), results determination and calculation, other information processing and communications.

Where genetic analysis is of concern, the system sensor chip and/or magnetic bead will be designed so that the specific genetic sequences may be easily altered to comprise particular market applications such as disease panels and forensic sampling.

Examples of application included human medical diagnostics. One application of this is in vitro diagnostics (IVD). In the US there are 5,200 hospital and commercial labs and 89,000 physician's offices labs (POLS) that perform clinical diagnostic tests. Both the laboratories and POLs will benefit from the further expansion of genetic based tests.

Another area of application is agriculture and animal husbandry wherein the use of the current invention may help to accelerate the process of selective breeding in both plants and animals. The system may also be used to identify the presence of infectious organisms in livestock and feed lots.

The foregoing is intended to be illustrative of the embodiments of the present invention, and are not intended to limit the invention in any way. Although the invention has been described with respect to specific modifications, the details thereof are not to be construed as limitations, for it will be apparent that various equivalents, changes and modifications may be resorted to without departing from the spirit and scope thereof and it is understood that such equivalent embodiments are to be included herein. All publications and patent applications are herein incorporated by reference to the same extent as if each individual publication or patent application was specifically and individually indicated to be incorporated by reference.

We claim:

1. A device for detecting a target molecule in a sample solution comprising: magnetic beads having in contact therewith polynucleotides capable of participating in an anchored strand displacement amplification reaction; a flow cell having channels for receiving a flowable medium, said flow cell further having at least one micro sensor comprising sensor pads and electrodes associated with each sensor pad.

2. A device according to claim 1 wherein said polynucleotides on each bead comprise a population of polynucleotides, said population further comprising both cleavable and noncleavable single stranded polynucleotides, wherein said cleavable and noncleavable quality is determined with respect to nicking of said polynucleotides in said strand displacement amplification reaction.

3. A device according to claim 2 wherein said population of said single stranded polynucleotides comprises nucleic acid sequences that are capable of hybridizing to 5′ or 3′ sequence of a target nucleic acid.

4. A device according to claim 2 wherein said sensor comprises between 64 and 4096 individual sensor pads.

5. A device for detecting a target molecule in a sample solution comprising: magnetic beads having in contact therewith polynucleotide probess capable of hybridizing to a target nucleic acid sequence; a flow cell having channels for receiving a flowable medium, said flow cell further having at least one micro sensor comprising sensor pads, said pads further having in contact therewith polynucleotide probes cabable of participating in an anchored strand displacement amplification reaction.

6. A device according to claim 5 wherein said polynucleotides on each sensor comprise a population of polynucleotides, said population further comprising both cleavable and noncleavable single stranded polynucleotides, wherein said cleavable and noncleavable quality is determined with respect to nicking of said polynucleotides in said strand displacement amplification reaction.

7. A device according to claim 6 wherein said population of said single stranded polynucleotides comprises nucleic acid sequences that are capable of hybridizing to 5′ or 3′ sequence of a target nucleic acid.

8. A device according to claim 6 wherein said sensor comprises between 64 and 4096 individual sensor pads.

9. A method for detecting target molecules comprising:
a. mixing microbeads of claim 1 or 5 with a sample solution containing a least one target nucleic acid of interest;
b. contacting said target nucleic acid to either said microbeads or said sensor pads;
c. performing a strand displacement reaction on said target nucleic acid sequence;
d. contacting said microbeads following said reaction of (c) with a micro sensor;
e. binding said microbeads to said sensor; and
f. detecting the presence of said microbeads bound to said sensor.
